(19) United States
(12) Patent Application Publication
(10) Pub. No.: US 2004/0122362 A1
(43) Pub. Date: Jun. 24, 2004

(54) Pseudo aneurysm repair system

(76) Inventors: Russell A. Houser, Livermore, CA (US); Brad Sharp, Irvine, CA (US); Christopher J. White, New Orleans, LA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/659,721

(22) Filed: Sep. 9, 2003

Related U.S. Application Data
(60) Provisional application No. 60/409,806, filed on Sep. 10, 2002.

(51) Int. Cl. A61M 29/00

(52) U.S. Cl. 604/102.01

(57) ABSTRACT

A method for draining and sealing a pseudo aneurysm in which a guide wire is inserted over a needle. Then a multi-lumen catheter is inserted over the guide wire. The catheter is used to occlude the vessel puncture and inhibit the flow of fluids between the vessel and the pseudo aneurysm. The pseudo aneurysm is then aspirated and the vessel puncture is allowed to clot and heal.

### RELATED APPLICATION

This application claims the benefit of U.S. Provisional Application No. 60/409,806, filed Sep. 10, 2002.

### BACKGROUND OF THE INVENTION

1. Field of the Invention

This invention relates generally to medical devices and more particularly to devices and methods for accessing and draining a pseudo aneurysm and sealing the related blood vessel.

2. Description of the Related Art

A pseudo aneurysm may form whenever a break in a vessel allows blood to leak out of circulation into an area surrounding the blood vessel. Typically, the break is a small hole in an artery, and it may be caused by trauma or infection. Many medical therapeutic and diagnostic procedures involve the percutaneous introduction of instrumentation into a vein or artery. In each of those procedures, the closing and subsequent healing of the resultant vascular puncture is critical to the successful completion of the procedure. Not infrequently, the vascular puncture becomes infected or simply fails to properly heal. Several days after the procedure, the patient will have the severe pain and swelling associated with a pseudo aneurysm.

When a pseudo aneurysm forms, blood and other fluids pool up within the adjacent fibrous tissue and other structures surrounding the vessel. The pseudo aneurysm thus subjects the surrounding anatomy to increased pressure causing severe pain and often tissue damage. In addition, draining the pseudo aneurysm and sealing the vessel may be a risky or difficult procedure depending on the location of the pseudo aneurysm and the overlying and surrounding anatomy.

The prior art includes devices for closing an opening in a vessel when completing a surgical procedure. These devices, however, enter the vessel through the opening that needs to be closed. Because procedures using these devices are often performed during the underlying surgery, many of these devices rely upon the guide wires or catheters that were used in the primary surgical procedure still in place. These devices are designed to seal the vessel prior to the formation of a pseudo aneurysm. Thus, they do not address the problem of treating a pseudo aneurysm after it has formed. There exists a need for a device and method designed for sealing a vessel at any point after surgery, even days later when a painful pseudo aneurysm has formed. Furthermore, a need exists for a device and method designed for accessing and draining a pseudo aneurysm.

### SUMMARY OF THE INVENTION

The present invention addresses some if not all of these needs. The method of this invention can be used to drain a pseudo aneurysm and to seal a puncture in a blood vessel. In one application, the method comprises inserting an introducer needle percutaneously directly into a pseudo aneurysm sack, and then inserting a guide wire through the needle and into the pseudo aneurysm sack. The practitioner next removes the needle while leaving the guide wire in place. The method further comprises advancing a multi-lumen catheter along the guide wire and into the pseudo aneurysm sack by feeding the guide wire through a lumen of the catheter or a slot or groove in the catheter. In one application, the practitioner may use the catheter to inject contrast media into the pseudo aneurysm sack. The practitioner then advances the guide wire through the related puncture in the blood vessel and advances the catheter along the guide wire and through the puncture. The practitioner then inflates one or more balloons attached to the distal end of the catheter to occlude the puncture in the blood vessel and inhibit blood and other fluids from flowing between the blood vessel and the pseudo aneurysm. The method further comprises aspirating the pseudo aneurysm sack and injecting a coagulant to help clot the vessel puncture. Lastly, the practitioner deflates the one or more balloons and removes the balloon(s), catheter, and guide wire.

In an alternative application, the inventive method comprises treating the pseudo aneurysm through the vasculature whereby an introducer needle is inserted into the vasculature and a guidewire is inserted therethrough. The guidewire is then directed through the vasculature to the site adjacent the pseudo aneurysm and through the opening in the vessel wall to the pseudo aneurysm.

### BRIEF DESCRIPTION OF THE DRAWINGS

FIG. 1 illustrates one application of the present invention and more specifically illustrates the step of removing the needle.

FIG. 2 illustrates one application of the present invention and more specifically illustrates the catheter system in place with the balloons inflated to occlude a puncture in a blood vessel.

FIG. 3 illustrates a perspective view of one embodiment of the catheter system designed for use with applications of the present invention.

FIG. 4 illustrates an exploded view of one embodiment of a catheter system designed for use with applications of the present invention.

FIG. 5 illustrates an exploded view of an alternate embodiment of a catheter system designed for use with applications of the present invention.

FIG. 6 illustrates another application of the present invention and more specifically illustrates the step of advancing a catheter over a guide wire.

FIG. 7 further illustrates another application of the present invention and more specifically the step of advancing a catheter over a guide wire.

FIG. 8 illustrates an embodiment of a catheter system with a catheter comprising a shaped shaft profile designed for use with applications of the present invention.

FIG. 9 illustrates an embodiment of a catheter system with a catheter having balloons comprising a shaped profile designed for use with applications of the present invention.

### DETAILED DESCRIPTION OF THE PREFERRED EMBODIMENT

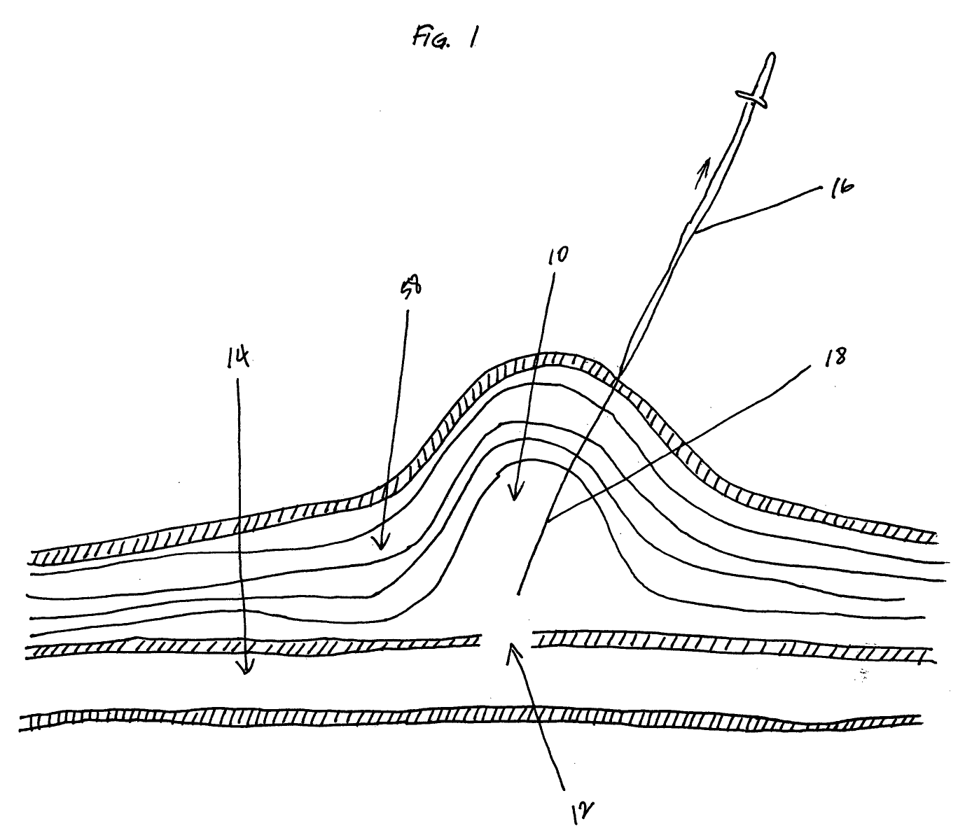

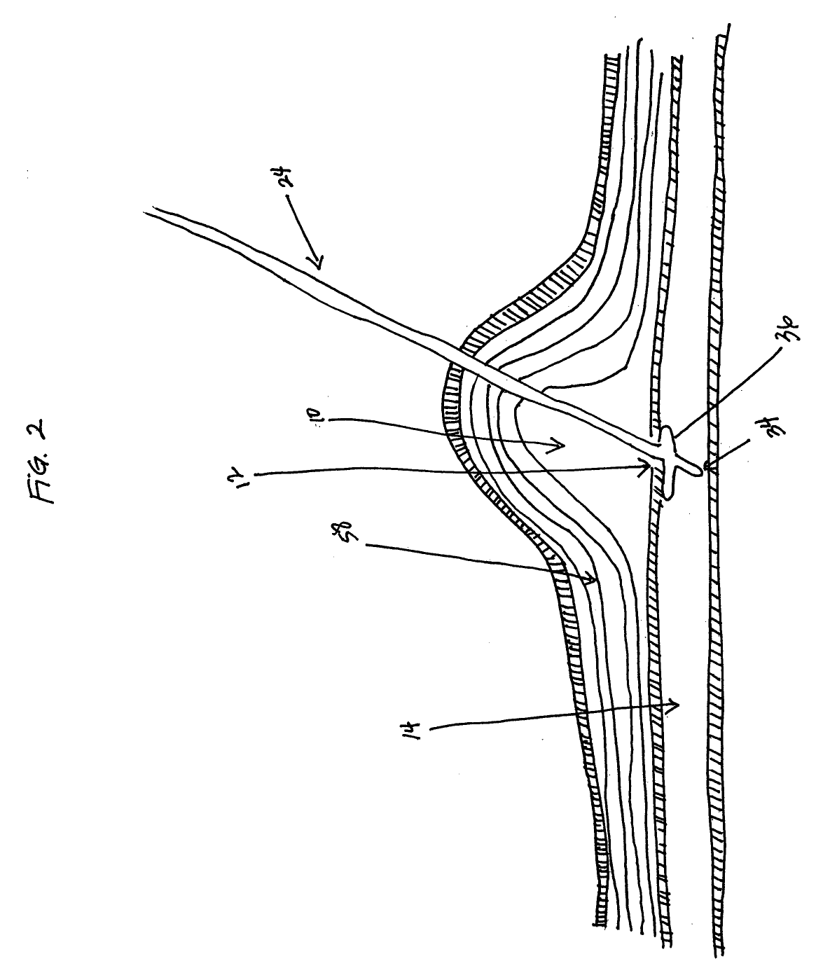

Referring to FIGS. 1 and 2, one application of the present invention comprises a method for draining a pseudo aneurysm 10 and sealing a related puncture 12 in a blood vessel 14. In this application, a practitioner inserts a needle 16 into the pseudo aneurysm sack 10. The practitioner can verify the location of the needle 16 in the pseudo aneurysm sack 10 by checking for red blood flow at the back of the needle 16. The method then comprises inserting a guide wire 18 through the needle 16 and into the pseudo aneurysm sack 10. In certain preferred embodiments, the guide wire 18 is about 0.01 inches, more preferably about 0.014 inches. The method further comprises removing the needle 16 while leaving the guide wire 18 in place. Next, the practitioner uses the guide wire 18 to insert a multi-lumen catheter system 20 designed for use with this application.

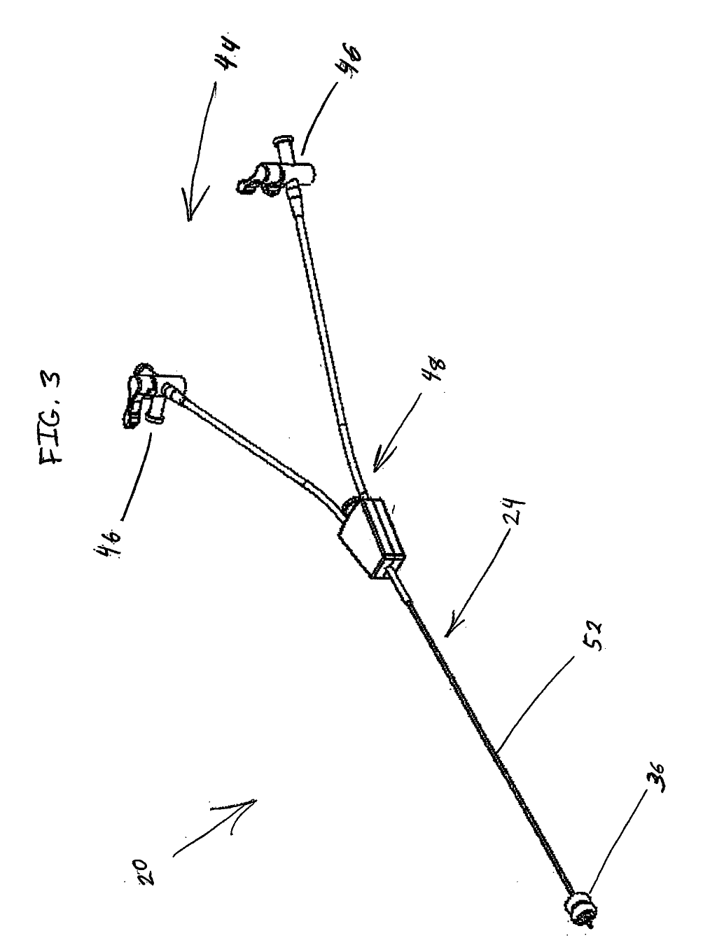

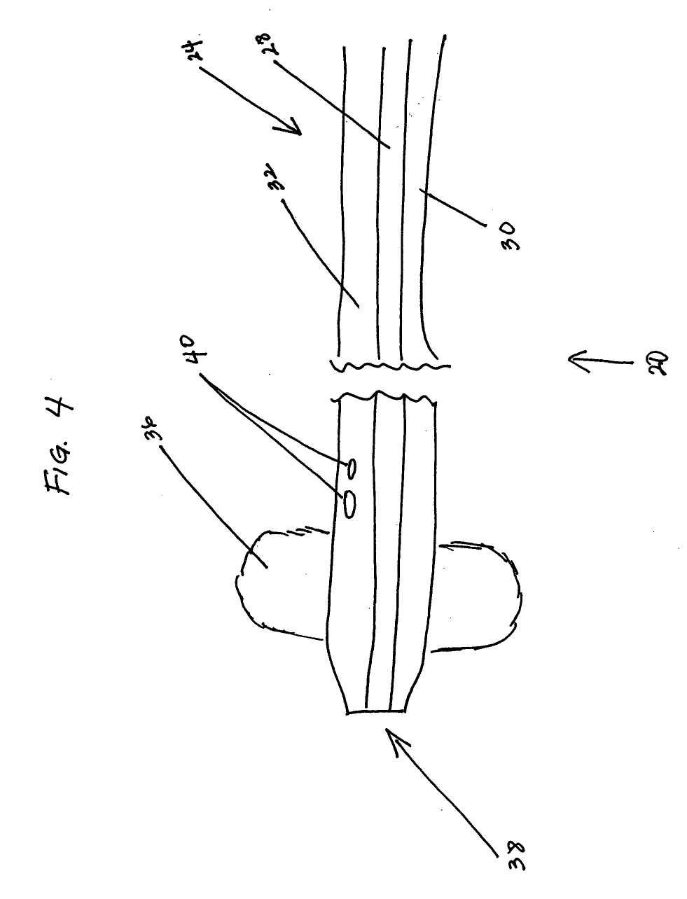

Referring to FIGS. 3 and 4, a multi-lumen catheter system 20 designed for use with this application of the present invention comprises a multi-lumen catheter 24 incorporating one lumen for a guide wire 28, one balloon inflation/deflation lumen 30, and one lumen for the withdrawal and infusion of fluids 32. The distal end 34 of the multi-lumen catheter 24 is preferably tapered to provide easier insertion through the skin. A balloon 36 is secured to the distal end 34 of the catheter 24. Proximate the balloon 36, either proximally or distally, the fluid lumen 32 includes one or more holes or slots 40. In one embodiment, the holes 40 are oval in shape along the longitudinal length of the catheter 24.

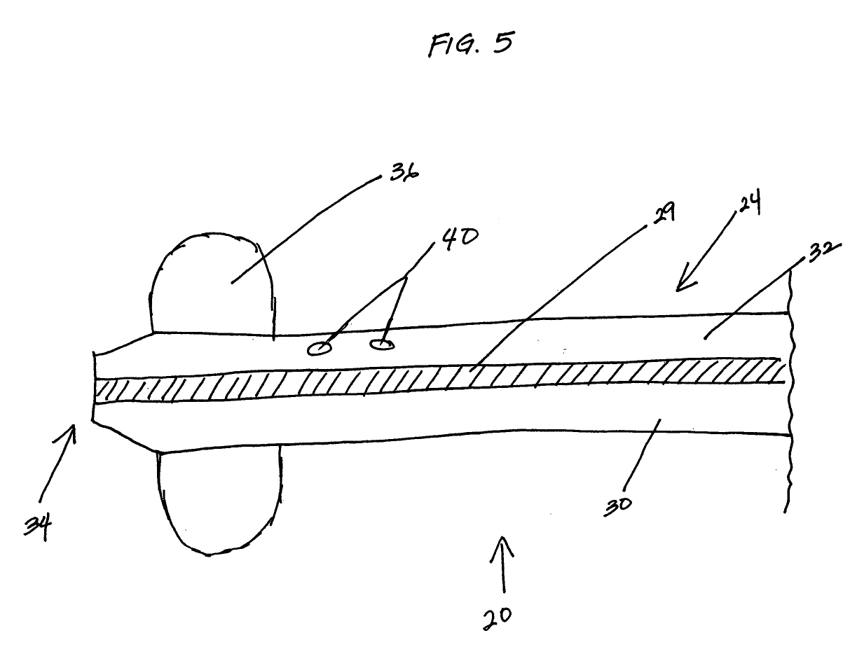

In the embodiment of FIG. 4, the three lumens are separate from each other. In an alternative embodiment, the lumens could be arranged coaxially. In addition, any of the three lumens can be designed for use as a guide wire lumen 28, a balloon inflation lumen 30, or a fluid lumen 32. The spatial relationship of the three lumens can vary. In an alternate embodiment shown in FIG. 5, the catheter 24 comprises a groove or slot 29 rather than a lumen for advancing the catheter 24 along the guide wire 18.

As shown in FIG. 3, the proximal end of the catheter 44 can include a tapered or twist Luer type fitting 46 connected to the balloon inflation/deflation lumen 30. The same type of fitting 46 can also be used for the proximal terminations of the guide wire lumen 28 and the fluid lumen 32. As shown in FIG. 3, these terminations may be incorporated into a single handle or yoke piece 48. In other embodiments, the terminations may have multiple individual ends. It will also be appreciated that any available fitting could be substituted for the Luer type fitting.

In some embodiments of the catheter system 20, the balloon 36 is a “T” or low profile disk shaped balloon. When this type of balloon is inflated to block an opening in a vessel, the top of the “T” or the disk can rest against the inside of the target vessel wall. Other embodiments of the catheter system may comprise more than one balloon, and the balloon(s) may have various different geometries including, for example, an hourglass or a peanut shape as illustrated in FIG. 3. An hourglass shaped balloon rests partially inside of the target vessel and partially outside of the target vessel. Other embodiments of the catheter system 20 include one or more tube balloons formed using a length of compliant material. With tube balloons, no secondary balloon forming or molding is required.

The balloon material can be compliant or non-compliant. In some embodiments, the balloon 36 may comprise one or more of the following compliant materials: polyethylene, polyurethane, or Tecoflex®. In some embodiments, the balloon may also comprise one or more of the following non-compliant materials: nylon, polyester (e.g. PET—polyethylene terephthalate), Pebax®, or polymide. The balloon may be fabricated using any suitable method or process including but not limited to molding, extrusion, heat forming, or cold forming. In addition, the balloon may be bonded to the catheter using any suitable method or process including, but not limited to, thermal, laser, adhesive, solvent, ultrasonic welding, or molding.

The tubular body 52 of the catheter 24 can be fabricated from any medical grade polymer such as nylon, polyethylene, Pebax®, polymide, polyester, polypropylene, and/or any combination of these or other suitable materials. In some embodiments, the shaft of the tubular body 52 can be reinforced using a polymer, a metal, a metal alloy (e.g., nitinol, stainless steel, Elgiloy®, iconel, 17-7PH®), and/or any combination of these or other suitable materials. In other embodiments, the shaft can be further reinforced, or separately reinforced, using a reinforcing component or technique including but not limited to the following: one or more wires having a round, flat, or any other cross sectional shape; multiple tubing layers, with or without a tie, or bonding layer; a mandrel; a hypo tube; irradiation; variable wall thickness; and/or any combination of these or other suitable reinforcing components and techniques. The reinforcing components can be either removable or fixed. In addition, the reinforcement can be along the entire length, or along a partial length at selected locations, and can be used to improve catheter trackability and pushability. The reinforcement can also provide a strain relief transition between bonded catheter segments. In some embodiments, the distal section of the tubular body 52 of the catheter 24 can be fabricated from a softer, more atraumatic material. The two or more sections may be joined together using heat and or adhesives or any other suitable process or method.

Methods of forming tubular bodies are known in the prior art. The tubular body 52 of the catheter 24 can be formed using any of the known methods of fabricating tubular bodies such as: single or multiple layer extrusion, casting, injection molding, dip coating, or any other suitable fabrication process or method. The tubular body 30 can also be reinforced by using a coil, braid, wrap, or any other suitable reinforcement. The holes or grooves used for fluid extraction or infusion ports 20 may be created by laser, drill sharpened mandrel, or any other suitable method or combination of methods.

The yoke piece 48 can be produced from polycarbonate, or another suitable material, using machining, casting, injection molding, or any other suitable process. In some embodiments, the yoke piece may contain interior grooves or channels to allow the catheter lumens to be joined or connected to the corresponding proximal tubes, providing fluid and air tight passages for each of the device lumens. During assembly, the two halves of the yoke piece can be bonded together using mechanical press fit, adhesives, solvents, sonic welding, or any other suitable method or process.

In one application of the present invention, the method comprises the step of advancing the guide wire 18 through the guide wire lumen of the catheter 28 until the holes 40 in the catheter 24 are inside of the pseudo aneurysm sack 10. It is contemplated that at least one way to load the guide wire is described in co-pending application No. Ser. 10/272,209, entitled “Guide Wire Insertion Tool,” which is incorporated herein by reference.

The method further comprises injecting contrast media that allows visualization of the pseudo aneurysm sack 10 and the location of the vessel puncture 12. In certain preferred applications, the contrast media is an ionic or non-ionic liquid contrast agent that can be visualized using a fluoroscope. Using the visualization, the practitioner then advances the guide wire 18 through the vessel puncture 12 and into the vessel 14.

Once the guide wire 18 is inside the vessel 14, the method further comprises advancing the catheter 24 along the guide wire 18 and through the vessel puncture 12. The distal end 34 of the catheter 24 should be far enough inside of the vessel 14 that the one or more balloons 36 can be inflated to occlude the vessel puncture as illustrated in FIG. 4. The method then comprises using the balloon inflation lumen 30 to inflate the one or more balloons 36 thereby isolating the vessel 14 from the pseudo aneurysm sack 10. In one application, the balloon(s) 36 can occlude the puncture from inside of the vessel 14. In other applications, a balloon 36 may be tapered in the middle so that half of the balloon occludes the puncture from inside the vessel 14 while the other half of the balloon occludes the puncture from outside of the vessel 14. Similarly in some applications with more than one balloon 36, one or more balloons may occlude the puncture from inside the vessel 14 while one or more balloons also occlude the pressure from outside the vessel 14. In still other applications, the balloon 36 may occlude the puncture only from outside the vessel 14.

The method further comprises using a syringe to aspirate the pseudo aneurysm sack 10 through the fluid lumen 32 of the catheter 24. In some applications, the practitioner may then use the fluid lumen 32 to inject a coagulant such as thrombin to help clot the puncture 12 in the vessel 14. The method then comprises deflating the one or more balloons 36 and slowly withdrawing the catheter 24 and the wire 18. Manual compression can help to stop any remaining bleeding.

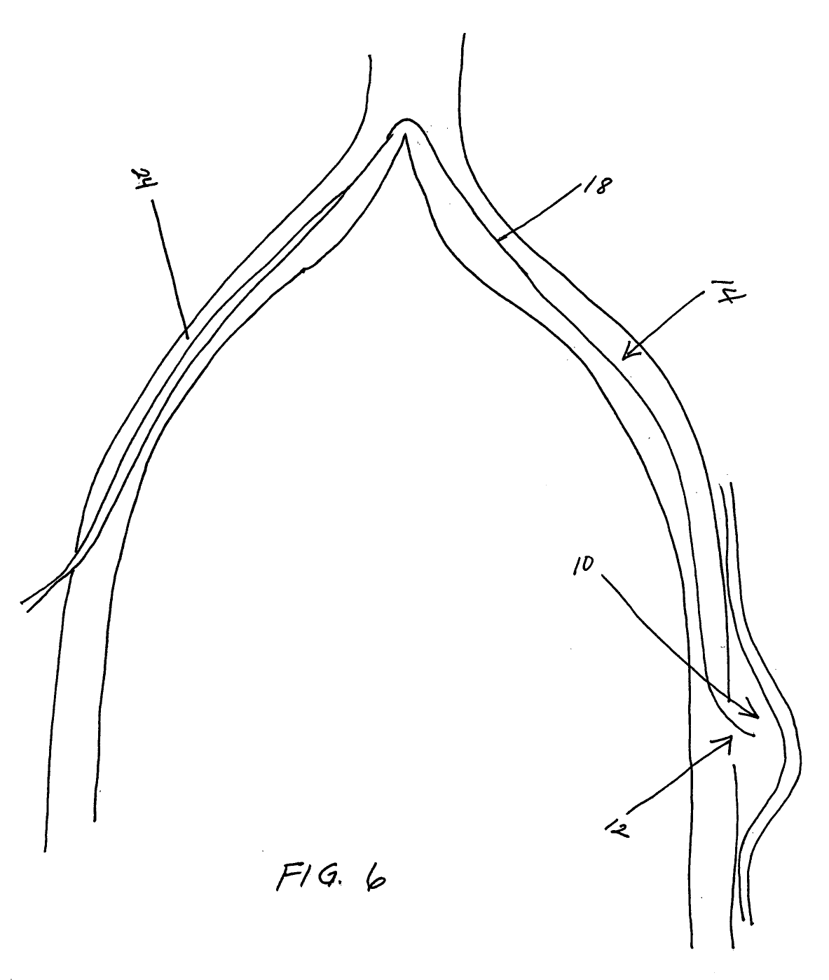

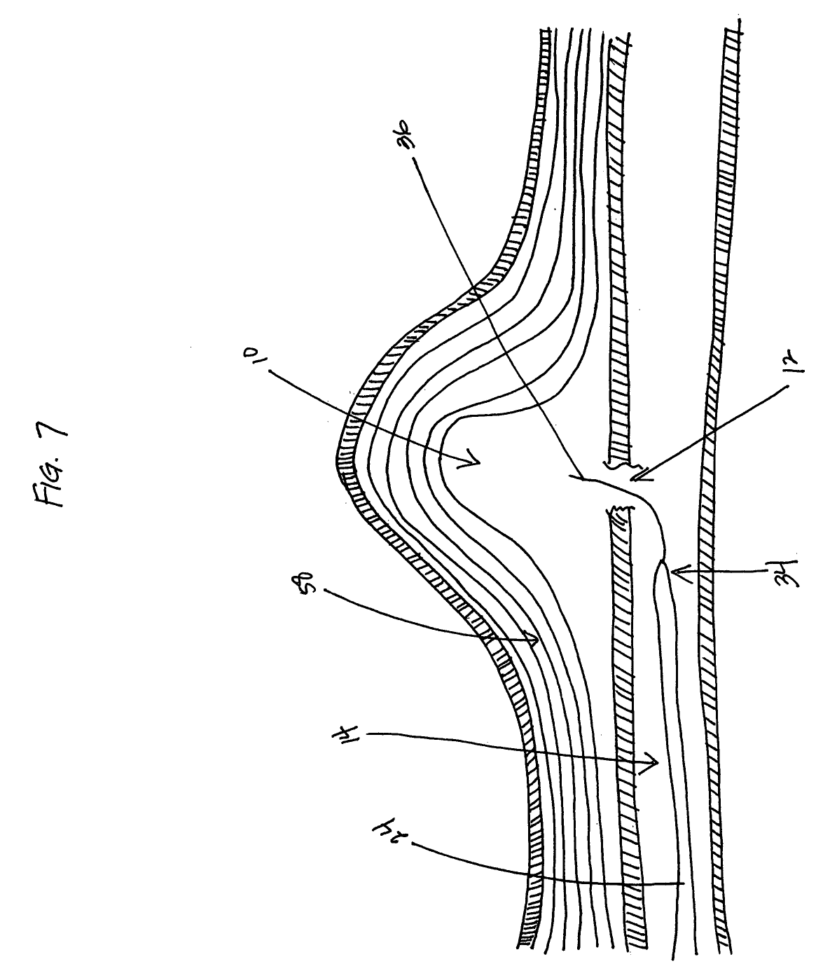

In another application of the present invention, shown in FIGS. 6 and 7, the method comprises accessing the pseudo aneurysm 10 from the vessel 14 rather than going through the anatomy surrounding the vessel 58. This application allows the practitioner to access the pseudo aneurysm 10 without disturbing the surrounding anatomy 58. In many cases, accessing the pseudo aneurysm 10 through the surrounding anatomy 58 can be a difficult, high risk, or painful procedure.

In this application, the method comprises inserting a needle 16 into a blood vessel 14 rather than into the pseudo aneurysm 10. As described above with respect to the first application, the method comprises inserting a guide wire 18 through the needle 16 and removing the needle 16 while leaving the guide wire 18 in place. The method next comprises advancing the catheter 24 into the vessel 14. Once the catheter 24 is inside of the vessel 14, the method comprises injecting a contrast media similar to the contrast media discussed above with regard to other applications. Using the contrast media to visualize the vessel 14, the practitioner advances the guide wire 18 along the vessel 14 to the location of the vessel puncture 12. The method further comprises advancing the guide wire 18 through the vessel puncture 12, and into the pseudo aneurysm sack 10. The method then comprises advancing the catheter 24 along the guide wire 18 such that the distal end 34 of the catheter 24 enters the pseudo aneurysm 10.

In this application, the method next comprises inflating the one or more balloons 36 using the balloon inflation lumen 30. The balloon(s) 36 occlude the vessel puncture 12 as discussed above. After inflating the balloon(s) 36 and occluding the vessel puncture 12, the practitioner drains the pseudo aneurysm sack 10 and seals the vessel puncture 12 as described above.

Although the application shown in FIG. 6 comprises approaching the pseudo aneurysm 10 through the femoral artery from the contra lateral side, other embodiments of the present invention comprise different approaches. For example, in other embodiments the practitioner may start in one artery and cross over to the relevant artery, or the practitioner may start in a vein and cross over to the relevant artery using a shunt. In addition, various embodiments of the present invention comprise starting at a point distal to the pseudo aneurysm 10 and heading proximally as well as starting at a point proximal to the pseudo aneurysm 10 and heading distally.

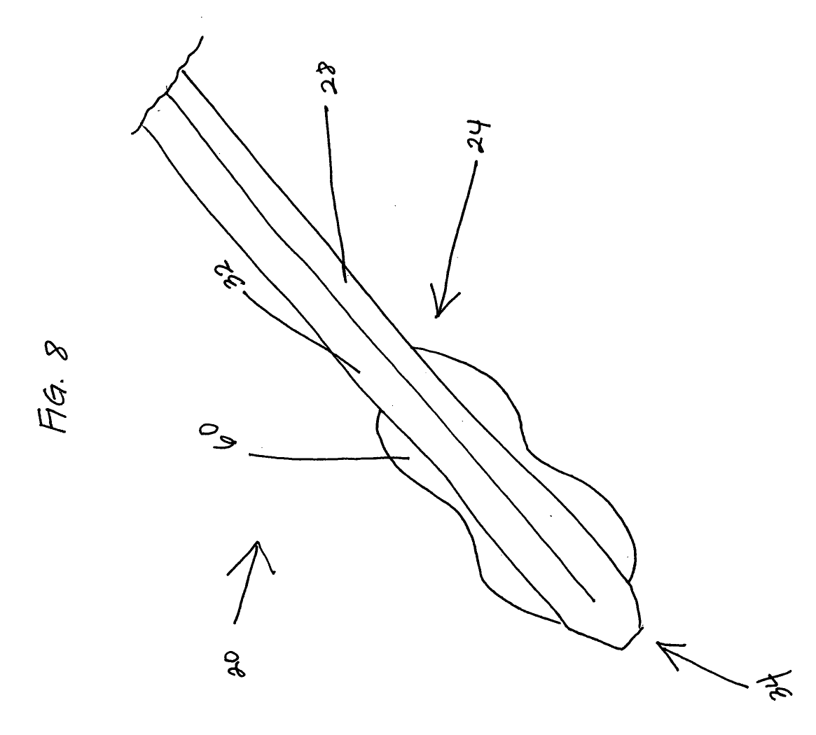

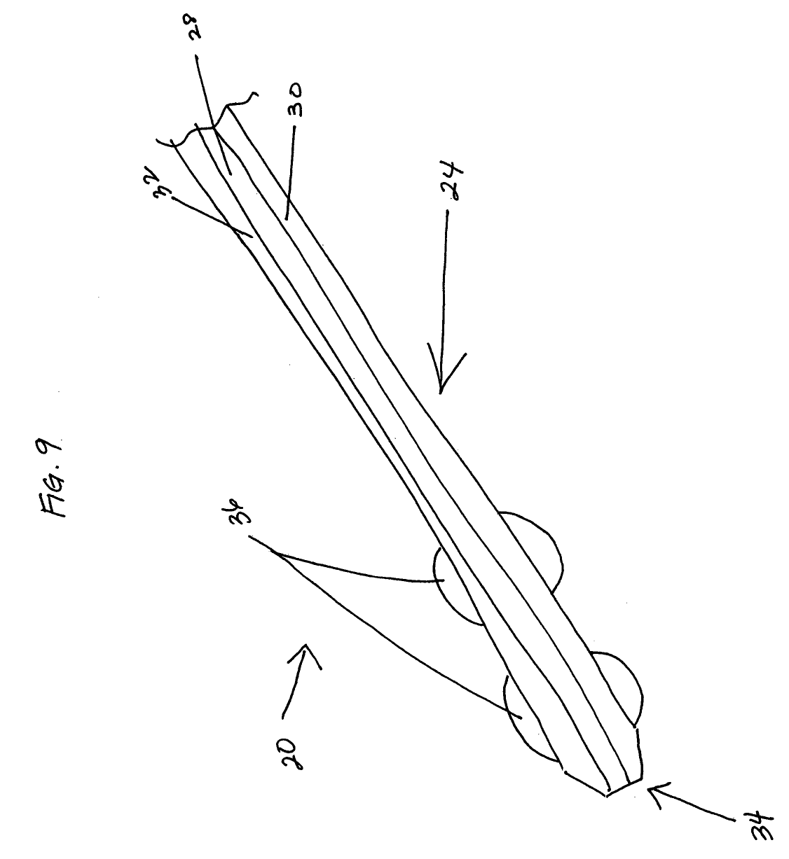

In yet another application of the present invention, the catheter system 20, as shown in FIG. 8, comprises a catheter 24 with an hourglass or peanut shaped shaft profile 60. This catheter system does not require a balloon 36; however, it may comprise one or more balloons. A practitioner can use this catheter system 20 with any of the applications described above. The practitioner can insert the catheter 24 directly percutaneously into the pseudo aneurysm 10, or the practitioner can insert the catheter into a blood vessel 14 and follow the vessel 14 into the pseudo aneurysm 10 as described above. The hourglass or peanut shaped shaft profile 60 can provide the practitioner with feedback as to the location of the distal end of the catheter 34 relative to the vessel puncture 12. The feedback comprises an increased resistance felt when pushing the larger diameter portions of the shaft through the puncture 12 and a decreased resistance felt when pushing the smaller diameter portion through the puncture. In an alternate application, a catheter 24 without an hourglass or peanut shaped profile can be used with an hourglass or peanut shaped balloon 36 near the distal end of the catheter 34. The practitioner can inflate the balloon 36 before inserting the catheter 24 through the vessel puncture 12. This can similarly provide the practitioner with feedback as to the location of the catheter 34 relative to the vessel puncture 12. Alternately two adjacent balloons 36 can provide the same effect as shown in FIG. 9.

In another application of the present invention, the catheter system 20 does not require a balloon 36 or an hourglass or peanut shaped shaft profile 60. In this application, the practitioner need not utilize a balloon 36 or a shaped shaft 60 to obstruct the vessel puncture 12. Instead, the method comprises injecting a material that is too viscous to easily seep into the vessel 14 through the puncture site 12. One such material is thixotropic fluid, but other materials may be used as well.

What is claimed is:

1. A method of draining a pseudo aneurysm sack and sealing an opening in the related blood vessel, said method comprising the steps of:
inserting a needle into a pseudo aneurysm sack;
advancing a guide wire through the needle;
advancing a catheter over the guide wire and into the pseudo aneurysm sack;
inflating one or more balloons to occlude a blood vessel puncture to inhibit fluid transfer between the blood vessel and the pseudo aneurysm sack;
aspirating fluids from the pseudo aneurysm sack; and
withdrawing the balloon, catheter, and guide wire.

2. The method of claim 1, further comprising the step of advancing the guide wire into a blood vessel.

3. The method of claim 1, further comprising the step advancing the catheter into a blood vessel.

4. The method of claim 1, wherein said blood vessel is an artery.

5. The method of claim 1, further comprising the step of injecting a coagulant to aid in clotting of a puncture in the blood vessel.

6. The method of claim 1, wherein said one or more balloons comprises a balloon having a disk shape.

7. The method of claim 1, wherein said one or more balloons comprises a balloon having a T shape.

8. The method of claim 1, wherein said one or more balloons comprises a balloon having an hourglass shape.

9. The method of claim 1, wherein said one or more balloons are inflated before the catheter is advanced through a puncture in the related blood vessel.

10. The method of claim 1, wherein said one or more balloons are inflated after the catheter is advanced through a puncture in the related blood vessel.

11. A method of draining a pseudo aneurysm and sealing the related blood vessel, said method comprising the steps of:
inserting a needle into a pseudo aneurysm sack;
advancing a guide wire through the needle;
advancing a catheter over the guide wire;
injecting one or more contrast media to aid in visualization of the pseudo aneurysm sack, the vessel, or a vessel puncture;
aspirating the pseudo aneurysm sack; and
withdrawing the catheter and guide wire.

12. The method of claim 11, further comprising the step of injecting a coagulant to aid in clotting of a puncture in the blood vessel.

13. The method of claim 11, wherein said blood vessel is an artery.

14. The method of claim 11, wherein said catheter further comprises one or more shaft reinforcing components.

15. The method of claim 14, wherein said shaft reinforcing component(s) extend along the entire length of the catheter.

16. The method of claim 14, wherein said shaft reinforcing component(s) extend along less than the entire length of the catheter.

17. The method of claim 14, wherein said shaft reinforcing components comprise a mandrel.

18. The method of claim 14, wherein said shaft reinforcing components comprise a hypo tube.

19. The method of claim 14, wherein said shaft reinforcing components comprise one or more wires.

20. The method of claim 14, wherein said shaft reinforcing components comprise one or more tubing layers.

21. The method of claim 14, wherein said catheter is reinforced by irradiation.

22. The method of claim 14, wherein said shaft reinforcing components comprise a catheter wall having variable thickness.

23. The method of claim 14, wherein said catheter comprises a distal section and a proximal section and wherein said distal section and said proximal section comprise different materials.

24. The method of claim 23, wherein the material of said distal section is more atraumatic than the material of said proximal section.

25. The method of claim 11, further comprising the step of injecting a material that is too viscous to seep through the vessel puncture.

26. The method of claim 11, wherein the catheter shaft comprises a shaped profile that can provide varying resistance as it is inserted through a vessel puncture.

27. The method of claim 26, wherein said shaped profile has an hour glass or peanut shape.

28. A method of draining a pseudo aneurysm and sealing the related blood vessel, said method comprising the steps of:
inserting a needle into a blood vessel;
advancing a guide wire through the needle;
inflating one or more balloons to occlude a blood vessel puncture to inhibit fluid transfer between the blood vessel and the pseudo aneurysm sack;
aspirating the pseudo aneurysm sack; and
withdrawing the balloon, catheter, and guide wire.

29. The method of claim 28, further comprising the step of advancing the guide wire through the blood vessel into a pseudo aneurysm sack.

30. The method of claim 28, further comprising the step of advancing a catheter over the guide wire and through the blood vessel into the pseudo aneurysm sack.

31. The method of claim 28, wherein said blood vessel is an artery.

32. The method of claim 28, further comprising the step of injecting a coagulant to aid in clotting of a puncture in the blood vessel.

33. The method of claim 28, wherein said one or more balloons comprises a balloon having a disk shape.

34. The method of claim 28, wherein said one or more balloons comprises a balloon having a T shape.

35. The method of claim 28, wherein said one or more balloons comprises a balloon having an hourglass shape.

36. The method of claim 28, wherein said one or more balloons are inflated before the catheter is advanced through a puncture in the related blood vessel.

37. The method of claim 28, wherein said one or more balloons are inflated after the catheter is advanced through a puncture in the related blood vessel.

38. A method of draining a pseudo aneurysm and sealing the related blood vessel, said method comprising the steps of:
inserting a needle into a blood vessel;
advancing a guide wire through the needle;
injecting one or more contrast media to aid in visualization of the pseudo aneurysm sack, the vessel, or a vessel puncture;
aspirating the pseudo aneurysm sack; and
withdrawing the catheter and guide wire.

39. The method of claim 38, wherein said blood vessel is an artery.

40. The method of claim 38, further comprising the step of injecting a coagulant to aid in clotting of a puncture in the blood vessel.

41. The method of claim 38, wherein said catheter further comprises one or more shaft reinforcing components.

42. The method of claim 41, wherein said shaft reinforcing component(s) extend along the entire length of the catheter.

43. The method of claim 41, wherein said shaft reinforcing component(s) extend along less than the entire length of the catheter.

44. The method of claim 41, wherein said shaft reinforcing components comprise a mandrel.

45. The method of claim 41, wherein said shaft reinforcing components comprise a hypo tube.

46. The method of claim 41, wherein said shaft reinforcing components comprise one or more wires.

47. The method of claim 41, wherein said shaft reinforcing components comprise one or more tubing layers.

48. The method of claim 41, wherein said catheter is reinforced by irradiation.

49. The method of claim 41, wherein said shaft reinforcing components comprise a catheter wall having variable thickness.

50. The method of claim 41, wherein said catheter comprises a distal section and a proximal section and wherein said distal section and said proximal section comprise different materials.

51. The method of claim 50, wherein the material of said distal section is more atraumatic than the material of said proximal section.

52. The method of claim 38, further comprising the step of injecting a material that is too viscous to seep through the vessel puncture.

53. The method of claim 38, wherein the catheter shaft comprises a shaped profile that can provide varying resistance as it is inserted through a vessel puncture.

54. The method of claim 53, wherein said shaped profile has an hour glass or peanut shape.

55. A device for draining and sealing a pseudo aneurysm comprising:
a multi-lumen catheter comprising at least one balloon inflation lumen and one fluid transfer lumen; and
one or more balloons attached to said catheter wherein said one or more balloons comprise a shaped profile that can provide varying resistance as it is inserted through a vessel puncture.

56. The device of claim 55, wherein said one or more balloons comprises an hourglass or peanut shaped balloon.

57. The device of claim 55, wherein said one or more balloons comprises two adjacent balloons.

58. A device for draining and sealing a pseudo aneurysm comprising:
a catheter comprising at least one fluid transfer lumen; and
a catheter shaft wherein said catheter shaft comprises a shaped profile that can provide varying resistance as it is inserted through a vessel puncture.

59. The device of claim 58, wherein said catheter shaft has an hourglass or peanut shape.

60. The device of claim 58, wherein said catheter further comprises a groove or slot for guiding the catheter along a guide wire.

61. The device of claim 58, wherein said catheter further comprises a guide wire lumen.

62. A device for draining and sealing a pseudo aneurysm comprising:
a catheter comprising at least one fluid transfer lumen wherein said fluid transfer lumen can be used for both withdrawal and infusion of fluids; and
a groove or slot for guiding the catheter along a guide wire.

63. The catheter of claim 60, further comprising a balloon inflation lumen and one or more balloons.

64. The catheter of claim 61, wherein said one or more balloons comprises a “T” or disk shaped balloon.

65. The catheter of claim 61, wherein said one or more balloons comprises a peanut or hourglass shaped balloon.

66. The catheter of claim 61, wherein said one or more balloons comprises two adjacent balloons.
